(19) United States
(12) Patent Application Publication
(10) Pub. No.: US 2023/0121091 A1
(43) Pub. Date: Apr. 20, 2023

(54) MICROORGANISM CONCENTRATION METHOD WITH ELASTIC POLYMERS

(71) Applicant: GIGABIOMOL BIYOTEKNOLOJI SANAYI VE TICARET LIMITED SIRKETI, Atasehir, Istanbul (TR)

(72) Inventors: Zühtü Tanil KOCAGÖZ, Atasehir, Istanbul (TR); Özge CAN, Atasehir, Istanbul (TR)

(21) Appl. No.: 17/048,829

(22) PCT Filed: Jun. 16, 2020
(86) PCT No.: PCT/TR2020/000003

§ 371 Date: Oct. 19, 2020

(30) Foreign Application Priority Data
Jun. 9, 2020 (TR) 2020/08937

(51) Int. Cl. G01N 1/40

(52) U.S. Cl. CPC G01N 1/4005; G01N 2001/4088

(57) ABSTRACT

Identification of infectious agents, in samples like blood, urine, mouthwash obtained from patients, is the most important tool for laboratory diagnosis of infectious diseases. Due to their technical nature, diagnostic tests can use only a small part of the sample obtained from the patient. For that reason, it is very important to concentrate infectious agents into a small volume of sample that will be used in diagnostic tests, to increase their sensitivity. Additionally, there may be substances that interfere by working of the diagnostic tests based on nucleic acid amplification like polymerase chain reaction (PCR). It is important to remove these substances so that this kind of diagnostic tests can work properly. This invention is a method of concentrating infectious agents in biological samples by using elastic polymer meshes. When added to liquid biological samples, these meshes remove water and small molecules from their environment, diminish the volume of the sample and thus enable concentrating the microorganisms Concentration of microorganism and removal of substances that inhibit the working of diagnostic methods, increase the sensitivity of these methods.

### FIELD OF THE INVENTION

The present invention is related to a method of concentrating infectious microorganisms in biological samples, by means of elastic polymer meshes, that remove small molecules from their environment, and the kits that will enable the application of this method. The present invention can be evaluated in the fields of biology and chemistry.

### THE BASIS OF THE INVENTION

Biological fluids from humans are often studied by various methods, for the diagnosis of infectious diseases. These methods include microscopy, culture, rapid antigen tests and nucleic acid amplification methods such as polymerase chain reaction (PCR). Regardless of the detection method, if the amount of the infectious agent in the sample is below the sensitivity limits of these tests, they cannot detect the agent. These tests, by nature, can evaluate a sample with a very small volume. However, many biological samples such as blood, urine and mouthwash can be easily taken tens of times more than the amount used in these tests. In addition, biological samples may contain substances that inhibit reactions that allow the detection of microorganisms, such as PCR, by amplifying their nucleic acids. The aim of this present invention is to concentrate infectious agents with elastic polymer molecular meshes, which reduce the volume of biological samples by removing water and other small molecules from the environment, as well as to eliminate small molecules that inhibit the reactions of diagnostic methods, thereby increasing the sensitivity of these tests.

### DETAILED DESCRIPTION OF THE INVENTION AND THE DIFFERENCE OF THE TECHNIQUE FROM KNOWN METHODS

Concentration of infectious agents in liquid biological samples increases the sensitivity of diagnostic methods.

The most commonly used method, to increase the detection sensitivity of infectious agents in liquid biological samples by diagnostic tests, is the concentration of microorganisms by centrifuging the liquids. After microorganisms are sedimented by centrifuging, the supernatant is poured and the organisms are concentrated in a small amount of liquid remaining at the bottom of the tube. The chance of detecting organisms such as bacteria and fungi, sought in the microscopic examination of this concentrated liquid, is higher compared to the original sample. It is also more likely to detect the organism by other diagnostic methods such as culture, molecular diagnostic methods and antigen tests, from a concentrated sample.

Centrifugation is a time-consuming process. It also requires a centrifuge with appropriate features. In laboratories without centrifuge, this concentration process cannot be applied. In order to concentrate the samples, it should be placed in the appropriate centrifuge tube, placed in the centrifuge device and generally rotated for several minutes. Then the supernatant is removed. There is a risk of transmission of organisms that cause disease, during these processes. Although the bacteria, fungi and parasites can be concentrated by centrifuging, the viruses cannot be concentrated directly by this way, as they are much smaller than these organisms.

Another method of concentrating infectious agents in biological fluids is by filtration. This process is used to concentrate microorganisms such as bacteria and fungi that cause contamination in foods such as milk. Filtration process requires special filters with 0.2-0.4 micron pore diameter. Since bacteria and fungi are larger than these pores, they cannot pass through the filter and are concentrated on the filter. Viruses, on the other hand, are very small and easily pass through such filter pores and cannot be condensed by filtration method. For this reason, both centrifugation and filtration are not suitable methods for condensing disease-causing viruses from biological liquid samples.

The present invention that we describe here is a method that removes water and other small molecules with elastic polymer meshes, reducing the volume of liquid medium, thereby concentrating infectious agents, including viruses, in biological samples. Three-dimensional cage-like structures made up of elastic polymers used in this method incorporate small molecules, while the volumes of the mesh structures expand. Meanwhile, the size of the pores on their surface allow organisms to concentrate in a small amount of fluid outside, by not allowing any infectious agents, including viruses, and their large antigens to enter this three-dimensional structure. The fluid containing the infectious agents outside these structures is taken from there and then used in diagnostic tests such as microscopy, culture, nucleic acid amplification and immunological tests. For example the sensitivity of nucleic acid amplification methods such as PCR, used to detect viral and bacterial agents such as SARS-CoV2 (new coronavirus), Influenza (flu virus), Mycobacterium tuberculosis, (tuberculosis bacillus), Streptococcus pyogenes can be increased as a result of concentration of infectious agents in mouthwash. Similarly sensitivity of antigen tests, microscopy and culture methods can also be increased. The sensitivity of the tests used in the diagnosis of microorganisms causing infections in central nervous system infection, can be increased by concentrating the cerebrospinal fluid and infectious agents causing urinary tract infections, by concentrating urine, using this new method. This new concentration method also contributes to the increased sensitivity of diagnostic tests, as molecules of small structure, which are present in biological fluids and inhibit the reactions of PCR and antigen tests, are also removed during the concentration process.

Regis Peytavi et al. in their patent application numbered US 8481265 B2 and named “Concentration and Enrichment of Microbial Cells and Microbial Nucleic Acids from Bodily Fluids” and in their equivalent applications made in other countries, stated that centrifugation or filtration methods are used for the concentration of microorganisms (Claim 14, item b). In this application the concentration method described is for bacteria and it is not possible to concentrate viruses by this method.

Apart from the classical filtration and centrifugation methods as described above, other microorganism concentration methods have been defined. These methods, which differ from the invention we have described above, and their different aspects, are described below.

In the patent application made by Kshirsagar, Manjiri T. et al. EP 2 205 717 B1 named “Microorganism Concentration Process”, and in their equivalent applications made in many countries, they describe a method of concentrating the microorganisms, by binding microorganisms to metal silicate crystals such as gamma-FO(OH) (iron oxide) and separating them from the liquid. In this method, it is not possible to reduce the liquid volume by removing water and other small molecules, thus concentrate microorganisms in a small volume, as we have described above in our new method. In patent application WO 2013/184186 named “Bismuth-containing Concentrating Agents for Microorganism” and similar applications made in various countries of the same application made by the same researchers, in order to concentrate microorganisms it has been proposed using bismuth salts instead of gamma-FO(OH). Also in their other application named “Microorganism concentration agent and method of making”—US 2014/001 1253 A1 they proposed using titanium dioxide, gold or platinum, bonded to silica sand for the binding of microorganisms, or in patent U.S. Pat. No. 9,575,059 B2 named “Lanthanum-based Concentration Agents” they proposed using lanthanum carbonate. In these applications, it is not possible to reduce the liquid volume by removing the small molecules by polymers as we defined in our invention in this document.

A similar patent application has been filed by Philip T. Feldsine to concentrate microorganisms. In patent application numbered US 2005/0123954 A1 and named as “Methods Compositions, and Kits for the concentration and detection of Microorganisms”, it is proposed that microorganisms are attached to various surfaces and concentrated by separating them from liquids. This is also not similar to the liquid concentration method with the elastic polymers that we described in this document.

None of the methods, we cited above concentrate viruses as the elastic polymer concentration method that we describe in this application. In patent document CNA031294081A, named “Protein, Fast Method Virus Concentration” a method based on co-precipitation of viruses bound to albumin is described. Similarly, in patent application U.S. Pat. No. 3,470,067 named “Concentration and Purification of Viruses from Particulate Magnetic Iron Oxide-Virus Complexes”, viruses are attached to magnetic iron oxide particles and concentrated. In the patent application CN 100509656C and named “Method for Concentrating Virus in Sewage or Sewage Treatment Plant Tail Water”, the concentration method defined is proposing binding of viruses to silica gel in the presence of Al³⁺ and separation after washing with sulfuric acid. All of these methods are based on concentration by binding viruses to a surface or by bonding and sedimentation together with various molecules, and they are different methods from volume reduction by removing small molecules from the environment with the elastic polymer meshes as we describe in this application. In the method we describe, microorganisms are not bound to any surface or molecule. Reducing the volume of liquid and concentrating viruses and other microorganisms is achieved by removal of the water and other small molecules surrounding the viruses that are freely present in the liquid, with elastic polymers.

In patent application CN103923883A named “Concentration and Purification Method for Influenza Virus” the concentration method of viruses defined is size exclusion chromatography, which is a different method than the method we describe in this application.

### EXAMPLES

1—Suspensions of bacteria, Escherichia coli, Staphylococcus aureus, Candida albicans and Mycobacterium tuberculosis H37Ra, were prepared in sterile 0.9% NaCl solution from fresh cultures. 10 ml of the suspensions were placed in a sterile tube, and previously synthesized and dried elastic polymers were added into the suspensions and waited for 5 minutes for concentration. 1/10 serial dilutions were made by using 100 microliters of unconcentrated and concentrated liquids, again using 0.9% NaCl. Then, 10 microliters of Escherichia coli and Staphylococcus aureus dilutions were inoculated to Mueller Hinton Agar, dilutions of Candida albicans to Sabouraud Dextrose Agar and Mycobacterium tuberculosis H37Ra dilutions to Löwenstein-Jensen media. Mueller Hinton and Sabouraud Dextrose Agar media were kept for 2 days and Löwenstein-Jensen media for 3 weeks in a 37° C. incubator. At the end of these periods, the number of colony forming units per milliliter of bacterial suspensions, were determined by counting the colonies formed in the media. An increase of 20 to 40-fold in samples concentrated by elastic polymers, relative to the dilute suspensions of all microorganisms tested was determined.

2—Samples that will be tested were prepared by adding SARS-CoV2 (new coronavirus) which were grown in Vero cells and than inactivated, into viral transport medium. In these samples, SARS-CoV2 RNA was investigated with Real-Time PCR before and after concentration by elastic polymers after extraction of RNA. It was identified that in samples concentrated with the polymer, SARS-CoV2 RNA was detected 4 to 6 cycles earlier, which indicated that the amount of virus in these samples was increased by 16 to 64 times.

We can conclude that this new method of concentration of microorganism including viruses with the elastic polymer meshes, has several advantages over previously described methods such as being a rapid, easy and effective method.

1- It is a method, that reduces the volume of fluid biological samples by removing water and other small molecules with molecular weight less than 500 Daltons, with elastic hydrophilic polymer meshes, thereby increasing the sensitivity of the diagnostic tests used in the detection of infectious agents and kits that depend on this method.

2- The “elastic polymer meshes” used in the method defined in claim 1, are characterized in that they are made of polyacrylamide or polyacrylic acid or polymethacrylate and its derivatives or dextran or agar or agarose or gelatin or glycogen or all kinds of other hydrophilic polymers.

3- The “diagnostic tests” used in the method defined in claim 1, are either microscopic examination, or culture or antigen and antibody detection by immunological tests including immunochromatographic tests and Enzyme Linked Immunosorbent Assay (ELISA), or nucleic acid amplification tests including polymerase chain reaction (PCR), loop-mediated amplification (LAMP), strand displacement amplification (SDA) and rolling circle amplification that are used in detection infectious agents.
